SEQUENCE LISTING (5 sequences)

SEQ ID NO: 1 - protein, 31 aa, Homo sapiens
HAEGTFTSDVSSYLEGQAAKEFIAWLVKGRG

SEQ ID NO: 2 - protein, 33 aa, homo sapien
HADGSFSDEMNTILDNLAARDFINWLIQTKITD

SEQ ID NO: 3 - protein, 39 aa, Gila monster
HGEGTFTSDLSKQMEEEAVRLFIEWLKNGGPSSGAPPPS

SEQ ID NO: 4 - protein, 39 aa, Gila monster
HSDGTFTSDLSKQMEEEAVRLFIEWLKNGGPSSGAPPPS

SEQ ID NO: 5 - protein, 44 aa, artificial sequence
  synthetic
HGEGTFTSDLSKQMEEEAVRLFIEWLKNGGPSSGAPPSKKKKKK

(19) United States
(12) Patent Application Publication
(10) Pub. No.: US 2010/0190715 A1
(43) Pub. Date: Jul. 29, 2010

(54) Stable Formulations of Peptides

(75) Inventors: Morten Schlein, Vaerlose (DK); Svend Ludvigsen, Lynge (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/752,634

(22) Filed: Apr. 1, 2010

Related U.S. Application Data
(63) Continuation of application No. 11/365,274, filed on Mar. 1, 2006.
Application No. 11/365,274 is a continuation of application No. PCT/DK04/00576, filed on Aug. 31, 2004.
(60) Provisional application No. 60/501,157, filed on Sep. 8, 2003.

(30) Foreign Application Priority Data
Sep. 1, 2003 (DK) PA 2003 01239

(51) Int. Cl. A61K 38/26; A61P 3/10

(52) U.S. Cl. 514/12

(57) ABSTRACT

Method for increasing the shelf-life of a pharmaceutical formulation comprising a glucagon-like peptide.

### CROSS-REFERENCE TO RELATED APPLICATIONS

This application is a continuation of U.S. application Ser. No. 11/365,274, filed Mar. 1, 2006, which is a continuation of International Application No. PCT/DK2004/000576, filed Aug. 31, 2004, which claims priority from Danish Patent Application No. PA 2003 01239 filed Sep. 1, 2003 and to U.S. Patent Application No. 60/501,157 filed Sep. 8, 2003.

### FIELD OF THE INVENTION

The present invention relates to the field of pharmaceutical formulations. More specifically the invention pertains to soluble and stable pharmaceutical formulations.

### BACKGROUND OF THE INVENTION

Therapeutic peptides are widely used in medical practise. Pharmaceutical compositions of such therapeutic peptides are required to have a shelf life of several years in order to be suitable for common use. However, peptide compositions are inherently unstable due to sensitivity towards chemical and physical degradation. Chemical degradation involves change of covalent bonds, such as oxidation, hydrolysis, racemization or crosslinking. Physical degradation involves conformational changes relative to the native structure of the peptide, which may lead to aggregation, precipitation or adsorption to surfaces.

Glucagon has been used for decades in medical practise within diabetes and several glucagon-like peptides are being developed for various therapeutic indications. The preproglucagon gene encodes glucagon as well as glucagon-like peptide 1 (GLP-1) and glucagon-like peptide 2 (GLP-2). GLP-1 analogs and derivatives as well as the homologous lizard peptide, exendin-4, are being developed for the treatment of hyperglycemia within type 2 diabetes. GLP-2 are potentially useful in the treatment of gastrointestinal diseases. However, all these peptides encompassing 29-39 amino acids have a high degree of homology and they share a number of properties, notably their tendency to aggregate and formation of insoluble fibrils. This property seems to encompass a transition from a predominant alpha-helix conformation to beta-sheets (Blundell T. L. (1983) The conformation of glucagon. In: Lefébvre P. J. (Ed) Glucagon I. Springer Verlag, pp 37-55, Senderoff R. I. et al., J. Pharm. Sci. 87 (1998)183-189, WO 01/55213). Aggregation of the glucagon-like peptides are mainly seen when solutions of the peptides are stirred or shaken, at the interface between solution and gas phase (air), and at contact with hydrophobic surfaces such as Teflon®.

Thus, various excipients must often be added to pharmaceutical compositions of the glucagon-like peptides in order to improve their stability. Shelf life of liquid parenteral formulations of these peptides must be at least a year, preferably longer. The in-use period where the product may be transported and shaken daily at ambient temperature preferably should be several weeks. Thus, there is a need for pharmaceutical compositions of glucagon-like peptides which have improved stability.

We have unexpectedly found that soluble pharmaceutical formulations of glucagon-like peptides exhibit increased stability when the formulations contain low concentrations of salts and buffer.

### BRIEF DESCRIPTION OF THE DRAWINGS

FIG. 1. ThT fluorescence assay of fibril formation in acylated GLP-1 samples. The samples contain 6 mg/ml acylated GLP-1, 5 μM ThT dissolved in water and adjusted to the stated pH. Experimental conditions are described in “Examples”. Briefly, the samples were incubated at 40° C. and shaken with 960 rpm in an Ascent Fluoroskan fluorescence plate reader. All data points are from the same experiment (i.e. all samples are from the same microtiterplate) and are means of eight replica and shown with standard deviations as error bars. Furthermore, these values at 20 and 40 hours are tabulated for each sample.

FIG. 2. ThT fluorescence assay of fibril formation in acylated GLP-1 samples dissolved in 8 mM phosphate buffer and adjusted to the stated pH values. All other conditions as described in FIG. 1.

FIG. 3. ThT fluorescence assay of fibril formation in acylated GLP-1 samples dissolved in water or various concentrations of phosphate buffer and adjusted to the stated pH values. All other conditions as described in FIG. 1.

FIG. 4. ThT fluorescence assay of fibril formation in acylated GLP-1 samples dissolved in various buffers with various pH. All other conditions as described in FIG. 1.

FIG. 5. ThT fluorescence assay of fibril formation in acylated GLP-1 samples dissolved in water or MOBS buffer and adjusted to the stated pH values. All other conditions as described in FIG. 1.

FIG. 6. ThT fluorescence assay of fibril formation in acylated GLP-1 samples dissolved in water or various buffers with various pH. All other conditions as described in FIG. 1.

FIG. 7. ThT fluorescence assay of fibril formation in acylated GLP-1 samples dissolved in Hepes buffers with various pH. All other conditions as described in FIG. 1.

FIG. 8. ThT fluorescence assay of fibril formation in acylated GLP-1 samples dissolved in water or histidine buffer with various pH. All other conditions as described in FIG. 1.

FIG. 9. ThT fluorescence assay of fibril formation in acylated GLP-1 samples dissolved in water pH 7.9 and with increasing concentration of NaCl. All other conditions as described in FIG. 1.

FIG. 10. Proton NMR spectrum of acylated GLP-1 with different additives at pH 7.9. N-terminal histidine signals of the imidazol sidechain are observed at approx 7.80 ppm and 7.00 ppm. The linewidth and position reflects the reduced flexibility of the N-terminal. NMR samples were prepared with 6 mg/ml acylated GLP-1 at pH 7.9 dissolved in 90%/10% H2O/D2O. NMR spectra were recorded at 600 MHz using a Varian Inova 600 MHz NMR instrument using 5 mm samples tubes. Sample volumes were 800 ul and spectra were measured at 27 degrees Celcius

FIG. 11. Amide protons of glu-9 (8.65 ppm) and gly-10 (8.42 ppm) under the same conditions as in FIG. 10. More intense amide proton signals reflect that these protons are relatively better shielded structurally from exchange with the water.

FIG. 12. Schematic representation of the time dependence of ThT fluorescence on fibril formation. The curve is Eq. (1) fitted to theoretically data points. The graphical meaning of lag time and kₐₚₚ are shown.

### DEFINITIONS

The following is a detailed definition of the terms used in the specification.

The term “soluble” as used herein referring to a formulation means a liquid formulation wherein substantially all of the active ingredient is on a soluble form. Thus soluble formulations typically are optically clear.

The term “shelf-stable” as used herein referring to a formulation means that the formulation remains suitable for its intended medical use until its expiration date. Parenteral liquid formulations typically must have a long shelf-life due to the distribution and stockpile before the product reaches doctors and patients. Typically shelf-life of liquid parenteral formulations of peptides are more than 1 year, more than 3 years, such as 5 years at the prescribed condition for keeping the product.

The term “buffer” as used herein means a chemical compound added to a formulation in order to prevent pH from changing over time.

The term “salts” as used herein means compounds formed, together with water, by reaction of an acid with a metallic base.

The term “effective amount” as used herein means a dosage which is sufficient to be effective for the treatment of the patient compared with no treatment.

The term “therapeutically effective concentration” as used herein means a concentration which renders treatment effective applying volumes of the pharmaceutical formulation which are typical in the art, e.g. 5 mL, 1 mL or lower than 500 μL.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “glucagon-like peptide” (GLP) as used herein refers to the homologous peptides, glucagon-like peptide 1 (GLP-1), glucagon-like peptide 2 (GLP-2), derived from the preproglucagon gene, exendins and analogues and derivatives thereof. The exendins which are found in the Gila monster are homologous to GLP-1 and also exert an insulinotropic effect. Examples of exendins are exendin-4 and exendin-3.

The glucagon-like peptides have the following sequences:

| | |
|---|---|
| | 1 5 10 15 20 25 30 35 |
| GLP-1 | HAEGT FTSDV SSYLE GQAAK EFIAW LVKGR G |
| GLP-2 | HADGS FSDEM NTILD NLAAR DFINW LIQTK lTD |
| Exendin-4 | HGEGT FTSDL SKQME EEAVR LEIEW LKNGG PSSGA PPPS-NH2 |
| Exendin-3 | HSDGT FTSDL SKQME EEAVR LEIEW LKNGG PSSGA PPPS-NH2 |

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. Two different and simple systems are often used to describe analogues: For example Arg³⁴-GLP-1(7-37) or K34R-GLP-1(7-37) designates a GLP-1 analogue wherein amino acid residues at position 1-6 have been deleted, and the naturally occurring lysine at position 34 has been substituted with arginine (standard single letter abbreviation for amino acids used according to IUPAC-IUB nomenclature).

The term “derivative” as used herein in relation to a parent peptide means a chemically modified parent protein or an analogue thereof, wherein at least one substituent is not present in the parent protein or an analogue thereof, i.e. a parent protein which has been covalently modified. Typical modifications are amides, carbohydrates, alkyl groups, acyl groups, esters, pegylations and the like. An examples of a derivative of GLP-1(7-37) is Arg³⁴, Lys²⁶(N^{ε}-(γ-Glu(N^{α}-hexadecanoyl)))-GLP-1(7-37).

The term “GLP-1 peptide” as used herein means GLP-1(7-37), a GLP-1 analogue, a GLP-1 derivative or a derivative of a GLP-1 analogue.

The term “GLP-2 peptide” as used herein means GLP-2(1-33), a GLP-2 analogue, a GLP-2 derivative or a derivative of a GLP-2 analogue.

The term “exendin-4 peptide” as used herein means exendin-4(1-39), an exendin-4 analogue, an exendin-4 derivative or a derivative of an exendin-4 analogue.

The term “stable exendin-4 compound” as used herein means a chemically modified exendin-4(1-39), i.e. an analogue or a derivative which exhibits an in vivo plasma elimination half-life of at least 10 hours in man, as determined by the following method. The method for determination of plasma elimination half-life of an exendin-4 compound in man is: The compound is dissolved in an isotonic buffer, pH 7.4, PBS or any other suitable buffer. The dose is injected peripherally, preferably in the abdominal or upper thigh. Blood samples for determination of active compound are taken at frequent intervals, and for a sufficient duration to cover the terminal elimination part (e.g. Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43(51):A143, 2000. Derived pharmacokinetic parameteres are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

The term “DPP-IV protected exendin-4 compound” as used herein means an exendin-4 compound which has been chemically modified to render said compound resistant to the plasma peptidase dipeptidyl aminopeptidase-4 (DPP-IV).

The term “immunomodulated exendin-4 compound” as used herein means an exendin-4 compound which is an analogue or a derivative of exendin-4(1-39) having a reduced immune response in humans as compared to exendin-4(1-39). The method for assessing the immune response is to measure the concentration of antibodies reactive to the exendin-4 compound after 4 weeks of treatment of the patient.

The term “isoelectric point” as used herein means the pH value where the overall net charge of a macromolecule such as a peptide is zero. In peptides there may be many charged groups, and at the isoelectric point the sum of all these charges is zero, i.e. the number of negative charges balances the number of positive charges. At a pH above the isoelectric point the overall net charge of the peptide will be negative, whereas at pH values below the isoelectric point the overall net charge of the peptide will be positive. The isoelectric point of a peptide may be determined by isoelectric focusing or it may be estimated from the sequence of the peptide by computational algorithms known in the art.

### DESCRIPTION OF THE INVENTION

In a first aspect the present invention relates to a soluble and shelf-stable pharmaceutical formulation comprising a therapeutically effective concentration of a glucagon-like peptide, a pharmaceutically acceptable preservative, a pharmaceutically acceptable tonicity modifier, optionally a pharmaceutically acceptable buffer, and a pH that is in the range from about 7.0 to about 8.0, characterized in that the content of salts is lower than about 5 mM, preferably lower than about 2 mM, even more preferable lower than about 1 mM.

In another aspect the present invention relates to a soluble and shelf-stable pharmaceutical formulation comprising a therapeutically effective concentration of a glucagon-like peptide, a pharmaceutically acceptable preservative, a pharmaceutically acceptable tonicity modifier, and a pH that is in the range from about 7.0 to about 8.0, characterized in that no buffer is present or low concentration of a buffer is present.

In one embodiment of the invention no buffer is present in the formulation.

In another embodiment of the invention substantially no buffer is present in the formulation.

In another embodiment of the invention a low concentration of buffer is present in the formulation.

In another embodiment of the invention the concentration of buffer in the formulation is less than about 8 mM, less than about 6 mM, or less than about 4 mM.

In another embodiment of the invention the buffer comprises no phosphorous.

In another embodiment of the invention the buffer is a zwitterion.

In another embodiment of the invention the buffer is glycyl-glycine.

In another embodiment of the invention the buffer is selected from the group consisting of HEPES, MOBS, MOPS and TES.

In another embodiment of the invention the buffer is histidine or bicine.

In another embodiment of the invention the tonicity modifier is not a salt.

In another embodiment of the invention the tonicity modifier is selected from the group consisting of glycerol, mannitol and dimethylsulphone.

In another embodiment of the invention the formulation has a pH in the range from about 7.4 to about 8.0

In another embodiment of the invention the formulation has a pH in the range from about 7.6 to about 7.9.

In another embodiment of the invention the isoelectric point of said glucagon-like peptide is from 3.0 to 7.0, preferably from 4.0 to 6.0.

In another embodiment of the invention the glucagon-like peptide is GLP-1, a GLP-1 analogue, a derivative of GLP-1 or a derivative of a GLP-1 analogue.

In another embodiment of the invention the GLP-1 analogue is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), and analogues thereof.

In another embodiment of the invention the derivative of a GLP-1 analogue is Arg³⁴, Lys²⁶(N^{ε}-(γ-Glu(N^{α}-hexadecanoyl)))-GLP-1(7-37).

In another embodiment of the invention the glucagon-like peptide is GLP-1, a GLP-1 analogue, a derivative of GLP-1 or a derivative of a GLP-1 analogue and the concentration in the pharmaceutical composition is higher than 1 mg/ml, preferably higher than 2 mg/ml, more preferred higher than 3 mg/ml, even more preferred higher than 5 mg/ml.

In another embodiment of the invention the concentration of the glucagon-like peptide in the pharmaceutical composition is in the range from about 1 mg/ml to about 25 mg/ml, preferably in the range from about 2 mg/ml to about 15 mg/ml, more preferred in the range from about 3 mg/ml to about 10 mg/ml, even more preferred in the range from about 5 mg/ml to about 8 mg/ml.

In another embodiment of the invention the glucagon-like peptide is exendin-4, an exendin-4 analogue, a derivative of exendin-4, or a derivative of an exendin-4 analogue.

In another embodiment of the invention the peptide is exendin-4.

In another embodiment of the invention the peptide is a stable exendin-4 compound.

In another embodiment of the invention the peptide is a DPP-IV protected exendin-4 compound.

In another embodiment of the invention the peptide is an immunomodulated exendin-4 compound.

In another embodiment of the invention the peptide is ZP10, i.e. HGEGTFTSDLSKQMEEEAVRLFIEWLKNGGPSSGAPPSKKKKKK-NH2.

In another embodiment of the invention the concentration of the glucagon-like peptide is exendin-4, an exendin-4 analogue, a derivative of exendin-4, or a derivative of an exendin-4 analogue in the pharmaceutical composition is from about 5 μg/mL to about 10 mg/mL, from about 5 μg/mL to about 5 mg/mL, from about 5 μg/mL to about 5 mg/mL, from about 0.1 mg/mL to about 3 mg/mL, or from about 0.2 mg/mL to about 1 mg/mL.

In another embodiment of the invention the glucagon-like peptide is GLP-2, a GLP-2 analogue, a derivative of GLP-2 or a derivative of a GLP-2 analogue.

In another embodiment of the invention the glucagon-like peptide is Gly²-GLP-2(1-33).

In another embodiment of the invention the derivative of GLP-2 or a derivative of a GLP-2 analogue has a lysine residue, such as one lysine, wherein a lipophilic substituent optionally via a spacer is attached to the epsilon amino group of said lysine.

In another embodiment of the invention the derivative of GLP-2 or said derivative of a GLP-2 analogue is an acylated GLP-2 compound.

In another embodiment of the invention the derivative of a GLP-2 analogue is Arg³⁰,Lys¹⁷(N^{ε}-(1-propyl-3-amino-hexadecanoyl)) GLP-2 (1-33).

In another embodiment of the invention the glucagon-like peptide is GLP-2, a GLP-2 analogue, a derivative of GLP-2 or a derivative of a GLP-2 analogue and the concentration of said glucagon-like peptide in the pharmaceutical composition is from 0.1 mg/mL to 100 mg/mL, from 0.1 mg/mL to 25 mg/mL, or from 1 mg/mL to 25 mg/mL.

In another embodiment of the invention the preservative is selected from phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof.

In another aspect the invention relates to a method for preparation of a pharmaceutical composition, comprising dissolving the GLP compound and admixing the preservative and tonicity modifier.

In another aspect the invention relates to a pharmaceutical formulation having a pH between about 7.4 to about 8.0, said composition comprising a glucagon-like peptide and at least one pharmaceutically acceptable excipient, wherein said composition is shelf stable as measured in a Thioflavin T assay as described herein which shows less than three fold increase of the Thioflavin T fluorescence from 20 hours to 40 hours during incubation of the sample at 40° C. (based on the mean Thioflavin T fluorescence at each time point).

In another aspect the invention relates to a pharmaceutical formulation having a pH between about 7.4 to about 8.0, said composition comprising a glucagon-like peptide and at least one pharmaceutically acceptable excipient, wherein said composition is shelf stable as measured in a Thioflavin T assay as described herein which shows less Thioflavin T fluorescence after storage of the composition for 40 hours at 40° C. than a similar formulation buffered by 8 mM phosphate at the same pH.

In another aspect the present invention relates to a method for the treatment of hyperglycemia comprising parenteral administration of an effective amount of the pharmaceutical composition comprising a GLP-1 peptide to a mammal in need of such treatment.

In another aspect the invention relates to a method for the treatment of obesity, beta-cell deficiency, IGT or dyslipideamia comprising parenteral administration of an effective amount of the pharmaceutical composition comprising a GLP-1 peptide to a mammal in need of such treatment.

In another aspect the present invention relates to a method for the treatment of short bowels syndrome comprising the administration of a formulation comprising a GLP-2 compound to a mammal in need of such treatment.

The use of excipients such as preservatives, isotonic agents and surfactants in pharmaceutical compositions is well-known to the skilled person. For convenience reference is made to Remington: The Science and Practice of Pharmacy, 19^{th} edition, 1995.

The parent glucagon-like peptide can be produced by peptide synthesis, e.g. solid phase peptide synthesis using t-Boc or F-Moc chemistry or other well established techniques. The parent glucagon-like peptide can also be produced by a method which comprises culturing a host cell containing a DNA sequence encoding the polypeptide and capable of expressing the polypeptide in a suitable nutrient medium under conditions permitting the expression of the peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of peptide in question.

The DNA sequence encoding the parent peptide may suitably be of genomic or cDNA origin, for instance obtained by preparing a genomic or cDNA library and screening for DNA sequences coding for all or part of the peptide by hybridisation using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the peptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The DNA sequence may also be prepared by polymerase chain reaction using specific primers, for instance as described in U.S. Pat. No. 4,683,202 or Saiki et al., Science 239 (1988), 487-491. The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the peptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art, cf. for instance Sambrook et al., supra.

The DNA sequence encoding the peptide may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracyclin, chloramphenicol, neomycin, hygromycin or methotrexate.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present peptide and includes bacteria, yeast, fungi and higher eukaryotic cells. Examples of suitable host cells well known and used in the art are, without limitation, E. coli, Saccharomyces cerevisiae, or mammalian BHK or CHO cell lines.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### EXAMPLES

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 309, 274-284].

The time course for fibril formation can be described by a sigmoidal curve with the following expression [Nielsen et al. (2001) Biochemistry 40, 6036-6046]:

$$F = {f}_{1} + {m}_{i} t + \frac{{f}_{f} + {m}_{f} t}{1 + {e}^{- [ ( t - {t}_{0} ) / τ ]}} \qquad Eq . ( 1 )$$

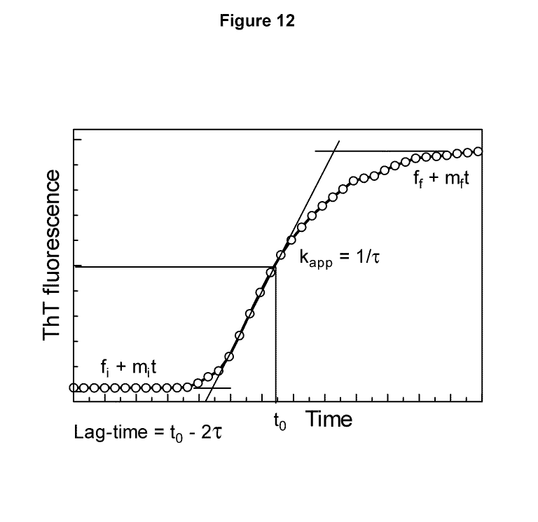

Here, F is the ThT fluorescence at the time t (see FIG. 12). The constant t₀ is the time needed to reach 50% of maximum fluorescence. The two important parameters describing fibril formation are the lag-time calculated by t₀−2τ and the apparent rate constant kₐₚₚ=1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

Based on a proposed mechanism for insulin fibrillation [Nielsen et al. (2001) Biochemistry 40, 6036-6046], one could hypothesize that fibrillation of acylated GLP-1 requires a fraction of molecules to dissociate into monomers, which further undergo partially unfolding. The physico-chemical properties of the solvent/solution may affect the degree of self-assembly as well as the flexibility and partial unfolding of acylated GLP-1 molecules, i.e. factors responsible for initiation of amyloid fibril formation.

Sample Preparation

Samples were prepared freshly before each assay. Usually, acylated GLP-1 was dissolved to 6 mg/ml in desired buffer or solvent. The pH of the sample was adjusted to the desired value using appropriate amounts of concentrated NaOH and HClO₄. Thioflavin T was added to the samples from a 1 mM stock solution in H₂O to a final concentration of 5 μM.

Sample aliquots of 100 μl were placed in a 96 well microtiter plate (Packard Opti-Plate™-96, white polystyrene). Usually, eight replica of each sample (corresponding to one test condition) was placed in one column of wells. The plate was sealed with Scotch Pad (Qiagen).

Incubation and Fluorescence Measurement

Incubation at given temperature, shaking and measurement of the ThT fluorescence emission were done in a Fluoroskan Ascent FL fluorescence platereader (Thermo Labsystems). Temperature setting is possible up till 45° C., but usually sat at 40° C. The orbital shaking is selectable up till 1200 rpm, but adjusted to 960 rpm with an amplitude of 1 mm in all the presented data. Fluorescence measurement was done using excitation through a 444 nm filter and measurement of emission through a 485 nm filter.

Each run was initiated by incubating the plate at the assay temperature for 10 min. The plate was measured each hour for typically 45 hours. Between each measurement, the plate was shaken and heated as adjusted.

Data Handling

The measurement points were saved in Microsoft Excel format for further processing and curve drawing and fitting was performed using GraphPad Prism. The background emission from ThT in the absence of fibrils was negligible. The data points are typically a mean of eight samples and shown with standard deviation error bars. Only data obtained in the same experiment (i.e. samples on the same plate) are presented in the same graph ensuring a relative measure of fibrillation between experiments.

The data set may be fitted to Eq. (1). However, since full sigmodial curves in this case are not usually achieved during the measurement time, the degree of fibrillation is expressed as ThT fluorescence at 20 and 40 hours, calculated as the mean of the eight samples and shown with the standard deviation.

Examples on the effect of solvent and buffer on the physical stability of acylated GLP-1

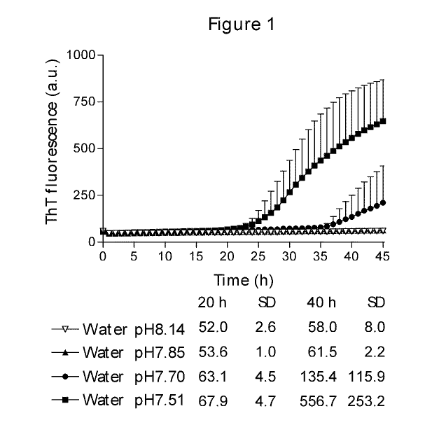

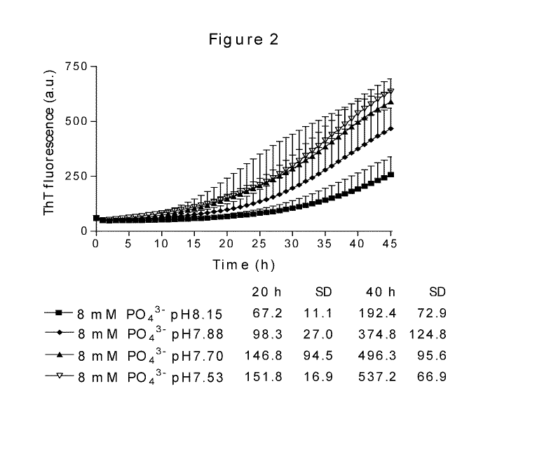

When acylated GLP-1 is dissolved in water rather than phosphate buffer, a significant increase in physical stability is surprisingly observed, compare FIG. 1 (acylated GLP-1 in water) with FIG. 2 (acylated GLP-1 in 8 mM phosphate buffer, these two figures show data from the same experiment). A general trend in all examples is shorter and shorter fibrillation lag-times when lowering pH in the interval pH 8.2 to pH 7.5. No significant fibrillation is observed after incubating acylated GLP-1 in water adjusted to pH8.14 or pH7.85 for 45 hours. In the presence of 8 mM phosphate, fibril formation is already observed at pH8.15. The physical stability is always better in water at a given pH compared to phosphate buffer. Using water adjusted to pH7.7 results in similar physical stability as achieved with 8 mM phosphate pH8.15, and in water at pH7.53 the physical stability is comparable with 8 mM phosphate pH7.88.

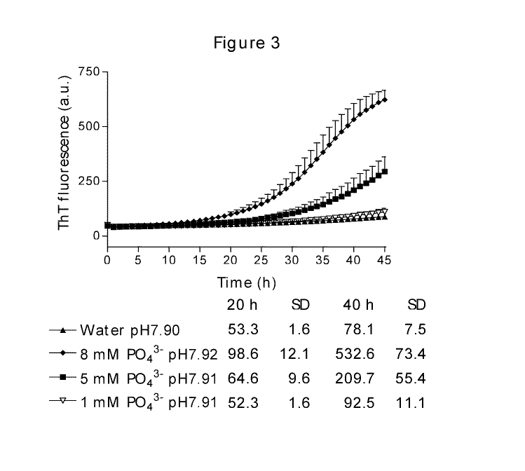

Less tendency to fibril formation is observed when lowering the phosphate concentration at a given pH. In FIG. 3, the phosphate concentration is gradually lowered from 8 mM phosphate to 1 mM phosphate at pH7.9. In 1 mM phosphate buffer, pH7.90, the physical stability is similar to that of an acylated GLP-1 solution in water, pH7.90.

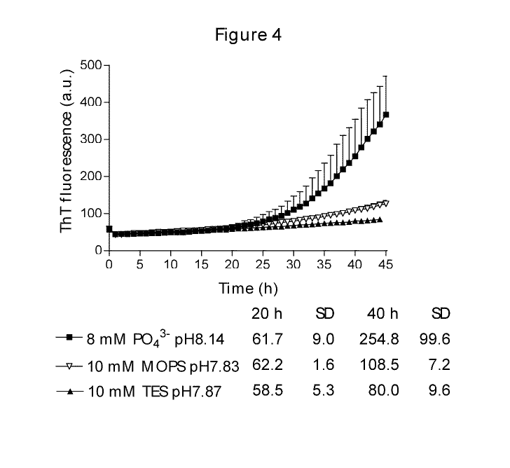

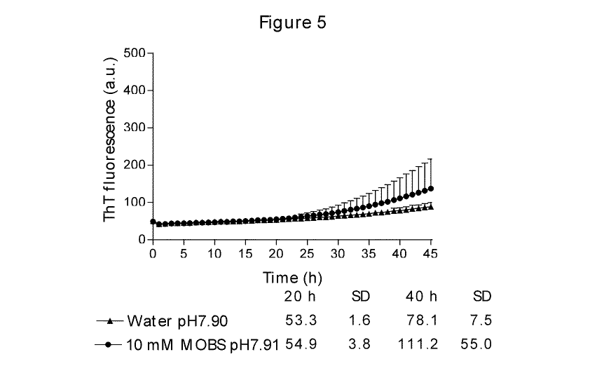

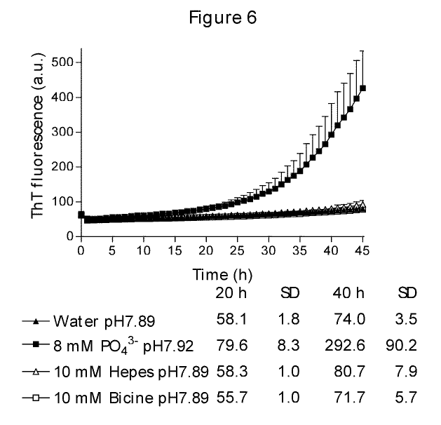

However, some zwitterionic buffer substances may be added without compromising the increased physical stability achieved in water in the absence of phosphate ions. Solutions of acylated GLP-1 with 10 mM MOPS or TES are both more physical stable than a solution in 8 mM phosphate at pH8.14, see FIG. 4, and 10 mM MOBS pH7.9 has similar physical stability as water pH7.9, see FIG. 5. Similar, HEPES or BICINE may be used as buffer at 10 mM without significantly increasing the fibrillation compared to water at pH7.89, see FIG. 6. In aqueous solutions, either non buffered or buffered with any of these two buffer substances, acylated GLP-1 are in all cases significantly more physically stable than in aqueous solution with 8 mM phosphate, pH7.92.

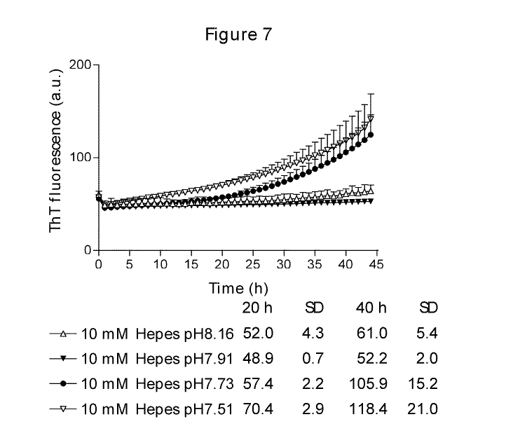

Buffered aqueous solutions using 10 mM HEPES become slightly more physically unstable when pH is gradually lowered from pH7.9 to pH7.51, see FIG. 7. However, these are still more stable than solutions buffered with phosphate, see FIG. 6. As with water, acylated GLP-1 in 10 mM HEPES pH7.73 is as physically stable as in 8 mM phosphate pH8.15, compare FIG. 7 with FIG. 2.

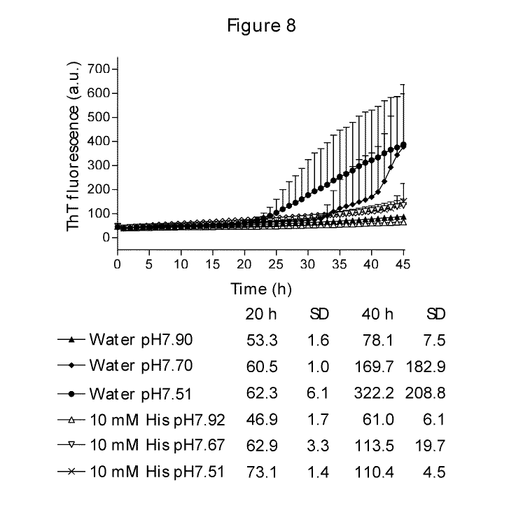

Some amino acids may also be used as zwitterionic buffers. Addition of 10 mM H is results in same physical stability of acylated GLP-1 as obtained in water at pH7.9, see FIG. 8. When lowering the pH, acylated GLP-1 dissolved in 10 mM His becomes surprisingly only marginally less stable, and at pH 7.5, acylated GLP-1 in 10 mM His is much more physically stable than acylated GLP-1 in non buffered aqueous solution pH7.5.

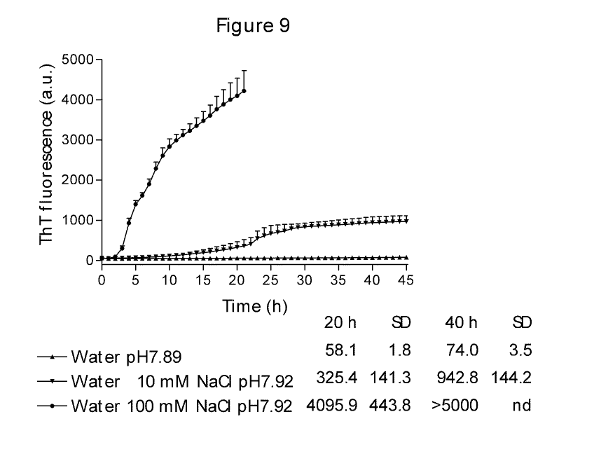

In addition to this, we claim that an added tonicity modifier must not be an electrolyte/salt. This is illustrated in FIG. 9, where the addition of 10 mM NaCl to an acylated GLP-1 solution in water promotes fibrillation. This effect is even more pronounced when adding 100 mM NaCl. However, both concentrations are much lower than the physiological NaCl concentration of 154 mM, usually applied when using NaCl as a tonicity modifier.

Examples on Solvent and Buffer Effects Observed with Nuclear Magnetic Resonance Spectroscopy (NMR)

Proton NMR spectroscopy of proteins in solutions has become a powerful technique to study structure and dynamics of a pure protein in solution (Wüthrich, K, “NMR of Proteins and Nucleic Acids”, (1986), ISBN 0-471-82893-9). Each proton (or proton group) in a protein will give rise to a resonance peak at a frequency which depends on the chemical and physical environment in the vicinity of each proton (or proton group) in the protein. A skilled artisan in the field of protein NMR spectroscopy will be able to assign most of the resonance peaks of a proton NMR spectrum of a protein to specific protons (or proton groups) in the protein given that the actual behavior of the protein in solution give rise to a well resolved proton NMR spectrum (well known to the skilled artisan in the field). The line width of the resonance peaks in the protein NMR spectrum reflects to some extend the size and dynamic properties of the protein in solution.

Aqueous solution of acylated GLP-1 at concentrations between 1 and 30 mg/ml, with or without buffers in the pH range 7.0 to 8.2 give rise to NMR spectra that to a high extend allow the afore mentioned resonance assignment. Thus several resonances of the NMR spectrum of this peptide can be assigned to specific atomic groups in the peptide. Recording NMR spectra of acylated GLP-1 under the conditions mentioned above but with different buffer substances in the solution allows a direct comparison of the structural and dynamical changes that acylated GLP-1 undergo as different buffers are applied.

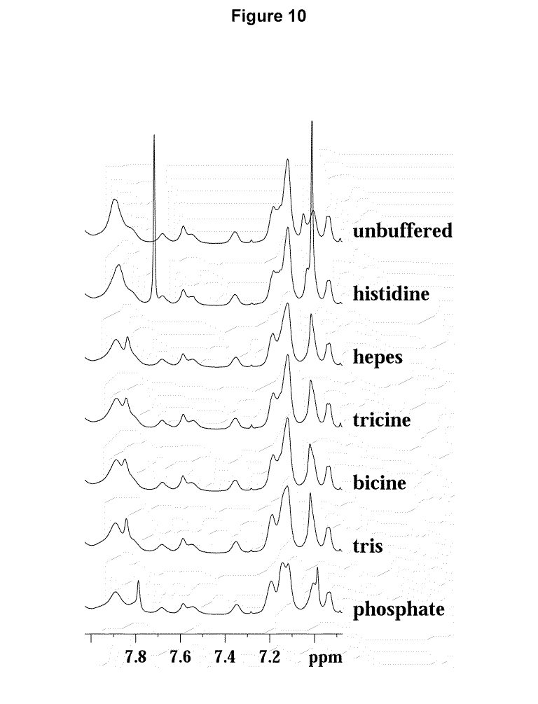

The proton resonances of the N-terminal histidine of acylated GLP-1 in aqueous solution are easily identified by the skilled artisan in the field, and both the resonance frequency and line width of these change surprisingly under the influence of different buffers or even absence of buffer. As buffers change from phosphate to tris, bicine, histidine, hepes the proton resonances of the imidazol side chain of the N-terminal histidine change according to FIG. 10. Narrow proton resonances of the imidazol side chain of the N-terminal histidine is reflecting a higher degree of structural flexibility of the N-terminal part of acylated GLP-1 in solution as compared to more broad proton resonances of the imidazol side chain of the N-terminal histidine which reflects that this part of the structure is more rigid and ordered.

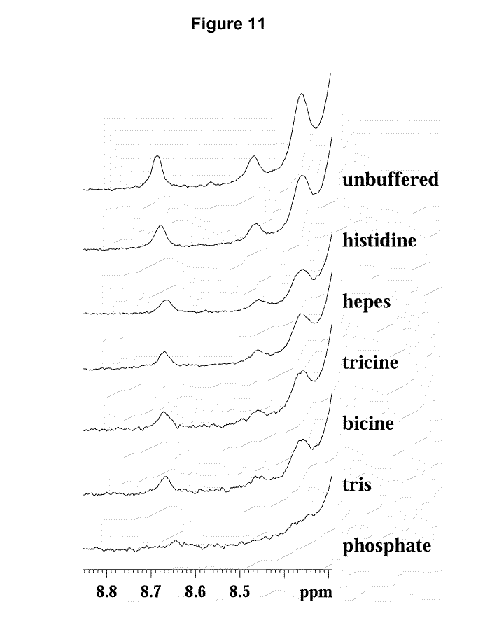

Additionally the amide protons of glutamic acid residue 9 and glycine residue 10 of acylated GLP-1 can be separately monitored under the influence of different buffer substances or variation in additives and their concentration under otherwise constant conditions. The exchange rate of amide protons in the pH range 7.0 to 8.2 reflects clearly the degree to which the specific amide protons are protected from direct access to the solvent water molecules. FIG. 11 shows that the two mentioned amide proton resonances vary in line width and intensity as buffers or additives are changed. Surprisingly, but very pronounced, is the almost disappearance of amides protons resonances in the NMR spectrum of acylated GLP-1 in aqueous solution buffered with 8 mM phosphate at pH 7.9 compared to the non-buffered situation. We can conclude that in aqueous solution without buffer substance the amide protons of glutamic acid residue 9 and amide proton of Glycine residue 10 are relatively more protected from the water molecules in solution as in the case using 8 mM phosphate buffer. This relative slower exchange of these two amide protons found in non buffered solution or solutions buffered with HEPES, bicine or histidine further emphasize that the N-terminal of acylated GLP-1 is more ordered and rigid under these latter conditions.

Most proton resonances of GLP-1 have a line width typical for proteins larger than one molecule of GLP-1. In depth interpretation of the proton NMR spectra of acylated GLP-1 shows that GLP-1 molecules bundle up in assemblies with several but well defined and limited number of acylated GLP-1 molecules almost constant in the concentration range 1 to 30 mg/ml. The change of the previously described resonances belonging to protons located in the N-terminal part of the acylated GLP-1 molecule could speculatively be explained by the ability of various buffers or their absence to let assemblies of acylated GLP-1 molecules pack more tightly thus providing additional explanation of the observed changes occurring in the proton NMR spectra of the peptide under variation of buffer substance or additive.

It is generally seen that relative slow amide proton exchange of N-terminal amino acids and more rigidity of the N-terminal part of the molecule is achieved under buffer free conditions or using HEPES, bicine, histidine buffers at pH 7.9 compared to phosphate buffer under otherwise constant conditions in aqueous solution at pH 7.90 of acylated GLP-1.

### Example on Making a Pharmaceutical Formulation

The compound (acylated GLP-1) was dissolved in a mixture of preservative (phenol), isotonic agent (mannitol, glycerol) and buffer (histidine, bicine, HEPES, MOPS, MOBS, TES or absence of buffer) to the desired concentration. The pH was adjusted to the specified value using Sodium Hydroxide and/or Hydrochloric Acid. Finally, the formulation was sterilized by filtration through a 0.22 μm sterile filter.

1. A soluble and shelf-stable pharmaceutical formulation, said formulation comprising a therapeutically effective concentration of a glucagon-like peptide, a pharmaceutically acceptable preservative, a pharmaceutically acceptable tonicity modifier that is not a salt, wherein said formulation does not comprise any buffer, and has a pH from 7.4 to 8.0.

2. A formulation according to claim 1, wherein said formulation has a salt concentration lower than about 5 mM.

3. A formulation according to claim 1, wherein the tonicity modifier is selected from the group consisting of glycerol, mannitol and dimethylsulphone.

4. A formulation according to claim 1, wherein the isoelectric point of said glucagon-like peptide is from 3.0 to 7.0.

5. A formulation according to claim 1, wherein said glucagon-like peptide is glucagon-like peptide 1 (GLP-1), a GLP-1 analogue, a derivative of GLP-1 or a derivative of a GLP-1 analogue.

6. A formulation according to claim 5, wherein the concentration of said glucagon-like peptide in the pharmaceutical composition is higher than 1 mg/ml.

7. A formulation according to claim 5, wherein the concentration of said glucagon-like peptide in the pharmaceutical composition is in the range from about 1 mg/ml to about 25 mg/ml.

8. A formulation according to claim 1, wherein said glucagon-like peptide is exendin-4, an exendin-4 analogue, a derivative of exendin-4, or a derivative of an exendin-4 analogue.

9. A formulation according to claim 8, wherein the concentration of said peptide in the pharmaceutical composition is from about 5 μg/mL to about 10 mg/mL.

10. A formulation according to claim 1, wherein said preservative is selected from phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof.

11. A method for preparation of a pharmaceutical formulation according to claim 1, said method comprising dissolving said GLP compound in water and admixing the preservative and tonicity modifier.

12. A pharmaceutical formulation having a pH between 7.4 to 8.0, said formulation comprising a glucagon-like peptide and at least one pharmaceutically acceptable excipient, wherein said formulation does not comprise any buffer, and is shelf stable as measured in a Thioflavin T assay which shows less than three fold increase of the Thioflavin T fluorescence from 20 hours to 40 hours during incubation of the sample at 40° C. than a similar formulation at the same pH and that comprises buffer.

13. A pharmaceutical formulation having a pH between about 7.6 to about 7.9, said formulation comprising a glucagon-like peptide and at least one pharmaceutically acceptable excipient, wherein said formulation does not comprise any buffer, and is shelf stable as measured in a Thioflavin T assay which shows less Thioflavin T fluorescence after storage of the composition for 40 hours at 40° C. than a similar formulation at the same pH and that comprises buffer.
